(19) United States
(12) Patent Application Publication
(10) Pub. No.: US 2022/0386961 A1
(43) Pub. Date: Dec. 8, 2022

(54) VASCULAR ACCESS DEVICES, SYSTEMS, AND METHODS FOR MONITORING PATIENT HEALTH

(71) Applicant: VERIS HEALTH INC., New York, NY (US)

(72) Inventors: James D. Mitchell, Walnut Creek, CA (US); Andrew Thoreson, Orono, MN (US)

(21) Appl. No.: 17/756,069

(22) PCT Filed: Nov. 18, 2020
(86) PCT No.: PCT/US2020/070803

§ 371 Date: May 16, 2022

Related U.S. Application Data
(60) Provisional application No. 62/937,139, filed on Nov. 18, 2019.

(51) Int. Cl. A61B 5/00; A61B 5/01

(52) U.S. Cl. CPC A61B 5/6852; A61B 5/7282; A61B 5/01; A61B 5/0004; A61B 5/746; A61B 5/6865

(57) ABSTRACT

Vascular access assemblies are disclosed herein. In one example, a vascular access assembly comprises a proximal catheter, a distal catheter, and a junction positioned between the proximal and distal catheters and configured to position the proximal and distal catheters in fluid communication. The junction may comprise one or more sensors configured to obtain physiological and/or operational measurements. The junction may be configured to wirelessly transmit the measurements to one or more local or remote computing devices for monitoring by a caregiver.

### CROSS-REFERENCE TO RELATED APPLICATION(S)

The present application claims the benefit of priority to U.S. Provisional Application No. 62/937,139, filed Nov. 18, 2019, which is incorporated by reference herein in its entirety.

### TECHNICAL FIELD

The present technology is generally directed to vascular access devices and associated systems and methods of use. In particular, the present technology is directed to vascular access devices and associated systems and methods for monitoring patient health.

### BACKGROUND

Vascular access devices encompass a variety of appliances deployed in either the arterial or venous space. As such, these devices require percutaneous passage into the vessel, and are either wholly or incompletely contained within the human corporis. Most commonly, the device is a tubular member constructed of one or more lumens of biocompatible materials, and is often accessible to, and in fluid communication with, medical tubing outside of the body. Such catheters are grouped into arterial or venous lines—venous lines further defined by location (i.e. peripheral, midline, and central). Conventional examples include the arterial line (“art-line”), peripheral intravenous line (IV), midline catheter, and central line, amongst others. These devices provide the conduit of various medicaments, fluids, and other agents, as well as the ability to sample the blood for diagnostic purposes. Since these devices are already required for most inpatient, and many outpatient, clinical applications, it would benefit to have the ability to provide more data of the patient than simply blood measurements. Using sensors for vital sign monitoring, response to therapy, and identification of patient decompensation with early intervention, could be incorporated into a minimally invasive device that is already required

### SUMMARY

The vascular access devices, systems, and methods of the present technology are configured to obtain patient physiological data while the vascular access device is placed within the patient, and determine one or more physiological parameters based on the measurements. The system may determine certain physiological parameters, for example, that indicate one or more symptoms of a health condition that requires immediate medical attention or hospitalization. Such physiological parameters can include those related to temperature, patient movement/activity level, heart rate, respiratory rate, blood oxygen saturation, and/or other suitable parameters described herein. Based on these parameters, the system may provide an indication to the patient and/or clinician that the patient has contracted or is at risk of contracting an adverse health condition.

The subject technology is illustrated, for example, according to various aspects described below, including with reference to FIGS. 1-10. Various examples of aspects of the subject technology are described as numbered clauses (1, 2, 3, etc.) for convenience. These are provided as examples and do not limit the subject technology.

1. A junction configured to be positioned between a fluid source or receptacle and a catheter, the catheter configured to be positioned in a blood vessel of a patient, wherein the junction comprises:

an inflow region;

an outflow region;

a sensor configured to obtain measurements; and

at least one controller configured to be communicatively coupled to the sensor,
wherein the at least one controller is further configured to—
obtain the measurements via the sensor while the catheter is positioned within the blood vessel; and
determine at least one physiological parameter based on the measurements.

2. The junction of Clause 1, wherein the junction is a portion of a central venous catheter, a midline catheter, a peripherally inserted central catheter, a peripheral intravenous line, or an arterial line catheter.

3. The junction of any one of the previous Clauses, wherein at least a portion of the sensor is exposed at an exterior surface of the junction such that, when the junction is positioned against a patient's skin, the sensor is in contact with the patient's skin.

4. The junction of any one of the previous Clauses, wherein the sensor comprises a plurality of sensors.

5. The junction of Clause 4, wherein each of the plurality of sensors have a portion that is exposed at an exterior surface of the junction such that, when the junction is positioned against a patient's skin, each of the sensors is in contact with the patient's skin.

6. The junction of Clause 4, wherein at least one of the sensors is disposed at an exterior surface of the junction and at least one of the sensors is disposed at an interior region of the junction.

7. The junction of any one of the previous Clauses, wherein the junction is a unitary body formed of a molded material.

8. The junction of any one of the previous Clauses, wherein the junction includes a first sensor adjacent the inflow region and a second sensor adjacent the outflow region.

9. The junction of any one of the previous Clauses, wherein the junction includes an opening at the inflow region configured to receive a portion of a tubular shaft therethrough.

10. The junction of Clause 9, wherein the tubular shaft is an extension tube.

11. The junction of any one of the previous Clauses, wherein the inflow region includes a receiving region surrounded by a body of the junction, wherein the receiving region is configured to permanently or detachably receive a portion of a tubular shaft therein.

12. The junction of Clause 11, wherein the tubular shaft is an extension tube.

13. The junction of any one of the previous Clauses, wherein the junction is configured to be extracorporeally positioned during use.

14. The junction of any one of the previous Clauses, wherein the at least one controller is configured to compare the at least one physiological parameter to a predetermined threshold.

15. The junction of Clause 14, wherein the at least one controller is configured to provide an indication of the patient's health based on the comparison.

16. The junction of Clause 14, wherein, based on the comparison, the controller is configured to provide an indication of a health condition of a patient, the health condition being at least one of sepsis, pulmonary embolism, metastatic spinal cord compression, anemia, dehydration/volume depletion, vomiting, pneumonia, congestive heart failure, kidney failure, volume overload, performance status, arrythmia, neutropenic fever, acute myocardial infarction, pain, opioid toxicity, hyperglycemic/diabetic ketoacidosis, hypoglycemia, hyperkalemia, hypercalcemia, hyponatremia, one or more brain metastases, superior vena cava syndrome, gastrointestinal hemorrhage, immunotherapy-induced or radiation pneumonitis, immunotherapy-induced colitis, diarrhea, cerebrovascular accident, stroke, pathological fracture, hemoptysis, hematemesis, medication-induced QT prolongation, heart block, tumor lysis syndrome, sickle cell anemia crisis, gastroparesis/cyclic vomiting syndrome, hemophilia, cystic fibrosis, chronic pain, and seizure.

17. The junction of any one of the previous Clauses, wherein the at least one controller is integrated with a body of the junction.

18. The junction of any one of the previous Clauses, wherein the at least one controller is further configured to transmit the measurements and/or the at least one physiological parameter to one or more remote computing devices.

19. The junction of any one of the previous Clauses, wherein the sensor is a pulse oximeter.

20. The junction of any one of the previous Clauses, wherein the sensor comprises at least one of a temperature sensor, a pressure sensor, a flow sensor, a moisture sensor, a biochemical sensor, or an impedance sensor.

21. A vascular access assembly (“VAA”), comprising:
an extension tube having a proximal end and a distal end;
a catheter having a proximal end and a distal end, the distal end configured to be positioned in a blood vessel of a patient;
a junction having an inflow region coupled to a distal end of the extension tube and an outflow region coupled to a proximal end of the catheter, wherein the junction is configured to fluidly couple the extension tube and the catheter;
a sensor configured to obtain measurements; and
at least one controller configured to be communicatively coupled to the sensor, wherein the at least one controller is further configured to—
   obtain the measurements via the sensor while the catheter is positioned within the blood vessel; and
   determine at least one physiological parameter based on the measurements.

22. The VAA of any one of the previous Clauses, wherein sensor is disposed at an interior region of the junction.

23. The VAA of any one of the previous Clauses, wherein the sensor is disposed at an exterior region of the junction.

24. The VAA of any one of the previous Clauses, wherein the sensor comprises a first sensor at an exterior region of the junction and a second sensor at an interior region of the junction.

25. The VAA of any one of the previous Clauses, wherein the sensor is disposed within a lumen of the extension tube.

26. The VAA of any one of the previous Clauses, wherein the sensor is disposed within a lumen of the catheter.

27. The VAA of any one of the previous Clauses, wherein the sensor is disposed within a wall of the catheter.

28. The VAA of any one of the previous Clauses, wherein the junction is a portion of a central venous catheter, a midline catheter, a peripherally inserted central catheter, a peripheral intravenous line, or an arterial line catheter.

29. The VAA of any one of the previous Clauses, wherein at least a portion of the sensor is exposed at an exterior surface of the junction such that, when the junction is positioned against a patient's skin, the sensor is in contact with the patient's skin.

30. The VAA of any one of the previous Clauses, wherein the sensor comprises a plurality of sensors.

31. The VAA of Clause 30, wherein each of the plurality of sensors have a portion that is exposed at an exterior surface of the junction such that, when the junction is positioned against a patient's skin, each of the sensors is in contact with the patient's skin.

32. The VAA of Clause 30, wherein at least one of the sensors is disposed at an exterior surface of the junction and at least one of the sensors is disposed at an interior region of the junction.

33. The VAA of any one of the previous Clauses, wherein the junction is a unitary body formed of a heat molded material.

34. The VAA of any one of the previous Clauses, wherein the junction includes a first sensor adjacent the inflow region and a second sensor adjacent the outflow region.

35. The VAA of any one of the previous Clauses, wherein the junction includes an opening at the inflow region configured to receive a portion of a tubular shaft therethrough.

36. The VAA of Clause 35, wherein the tubular shaft is an extension tube.

37. The VAA of any one of the previous Clauses, wherein the inflow region includes a receiving region surrounded by a body of the junction, wherein the receiving region is configured to permanently or detachably receive a portion of a tubular shaft therein.

38. The VAA of Clause 37, wherein the tubular shaft is an extension tube.

39. The VAA of any one of the previous Clauses, wherein the junction is configured to be extracorporeally positioned during use.

40. The VAA of any one of the previous Clauses, wherein the at least one controller is configured to compare the at least one physiological parameter to a predetermined threshold.

41. The VAA of Clause 40, wherein, the at least one controller is configured to provide an indication of the patient's health based on the comparison.

42. The VAA of Clause 40, wherein, based on the comparison, the controller is configured to provide an indication of a health condition of a patient, the health condition being at least one of sepsis, pulmonary embolism, metastatic spinal cord compression, anemia, dehydration/volume depletion, vomiting, pneumonia, congestive heart failure, kidney failure, volume overload, performance status, arrythmia, neutropenic fever, acute myocardial infarction, pain, opioid toxicity, hyperglycemic/diabetic ketoacidosis, hypoglycemia, hyperkalemia, hypercalcemia, hyponatremia, one or more brain metastases, superior vena cava syndrome, gastrointestinal hemorrhage, immunotherapy-induced or radiation pneumonitis, immunotherapy-induced colitis, diarrhea, cerebrovascular accident, stroke, pathological fracture, hemoptysis, hematemesis, medication-induced QT prolongation, heart block, tumor lysis syndrome, sickle cell anemia crisis, gastroparesis/cyclic vomiting syndrome, hemophilia, cystic fibrosis, chronic pain, and seizure.

43. The VAA of any one of the previous Clauses, wherein the at least one controller is integrated with a body of the junction.

44. The VAA of any one of the previous Clauses, wherein the at least one controller is integrated with the extension tube.

45. The VAA of any one of the previous Clauses, wherein the at least one controller is integrated with the catheter.

46. The VAA of any one of the previous Clauses, wherein the at least one controller is integrated with a portion of the VAA that is configured to be implanted within the patient during use.

47. The VAA of any one of the previous Clauses, wherein the at least one controller is integrated with a portion of the VAA that is configured to be positioned at a subcutaneous location during use.

48. The VAA of any one of the previous Clauses, wherein the at least one controller is integrated with a portion of the VAA that is configured to be positioned at an intravascular location during use.

49. The VAA of any one of the previous Clauses, wherein the at least one controller is integrated with a portion of the VAA that is extracorporeally positioned when the VAA is in use.

50. The VAA of any one of the previous Clauses, wherein the at least one controller is further configured to transmit the measurements and/or the at least one physiological parameter to one or more remote computing devices.

51. The VAA of any one of the previous Clauses, wherein the sensor is a pulse oximeter.

52. The VAA of any one of the previous Clauses, further comprising a fiber-optic member carried by the catheter.

53. The VAA of any one of the previous Clauses, wherein the sensor comprises at least one of a temperature sensor, a pressure sensor, a flow sensor, a moisture sensor, a biochemical sensor, or an impedance sensor.

54. The VAA of any one of the previous Clauses, wherein:
the at least one controller comprises a first controller and a second controller;
the first controller is integrated with a body of the junction and the second controller is separate from the body of the junction and not configured to be implanted within the patient; and
the first controller is in wireless communication with the second controller.

55. The VAA of Clause 54, wherein the first controller communicates with the second controller over at least one of a local area network and/or a personal area network.

56. The VAA of Clause 54, wherein the first controller communicates with the second controller via Bluetooth.

57. The VAA of Clause 54, wherein the first controller is remote from the second controller and communicates with the second controller via a wide area network.

58. The VAA of any one of Clauses 54 to 57, wherein the second controller is a smart device.

59. The VAA of any one of the previous Clauses, wherein:
the at least one controller comprises a first controller, a second controller, and a third controller;
the first controller is integrated with one or more of the extension tube, the junction, and the catheter;
the second controller is separate from the VAA and communicates with the first controller via a local area network and/or a personal area network;
the third controller is separate from the VAA and is one or more remote computing devices.

60. A method for monitoring the health of a patient, the method comprising:
obtaining physiological measurements of the patient via a sensor coupled to any one of the junctions of Clauses 1 to 20;
determining at least one physiological parameter based on the physiological measurements;
comparing the at least one physiological parameter to a predetermined threshold; and based on the comparison, providing an indication of the patient's health.

61. A method for monitoring the health of a patient, the method comprising:
obtaining physiological measurements of the patient via a sensor coupled to any one of the VAAs of Clauses 21 to 59,
determining at least one physiological parameter based on the physiological measurements;
comparing the at least one physiological parameter to a predetermined threshold; and
based on the comparison, providing an indication of the patient's health.

62. The method of Clause 60, wherein the sensing element comprises at least one of a temperature sensing element, a heart rate sensing element, a respiratory rate sensing element, a movement sensing element, a pressure sensing element, and an electrical signal sensing element.

63. The method of any one of the previous Clauses, wherein the at least one controller is configured to provide an indication that the patient is septic based on the comparison.

64. The method of any one of the previous Clauses, wherein the at least one physiological parameter is at least one of a temperature parameter, a heart rate parameter, a respiratory rate parameter, and an activity level parameter.

65. The method of any one of the previous Clauses, wherein the at least one physiological parameter is at least two of a temperature parameter, a heart rate parameter, a respiratory rate parameter, and an activity level parameter.

66. The method of any one of the previous Clauses, wherein:
the at least one physiological parameter comprises at least two of a temperature parameter, a heart rate parameter, a respiratory rate parameter, and an activity level parameter;
comparing the at least two physiological parameters to the predetermined threshold includes comparing each of the at least two physiological parameters to a corresponding predetermined threshold.

67. The method of any one of the previous Clauses, wherein:
the at least one physiological parameter comprises at least three of a temperature parameter, a heart rate parameter, a respiratory rate parameter, and an activity level parameter;
comparing the at least three physiological parameters to the predetermined threshold includes comparing each of the at least three physiological parameters to a corresponding predetermined threshold.

68. The method of any one of the previous Clauses, wherein:
the at least one physiological parameter comprises a temperature parameter, a heart rate parameter, a respiratory rate parameter, and an activity level parameter;
comparing the at least one physiological parameter to the predetermined threshold includes comparing the temperature parameter, the heart rate parameter, the respiratory rate parameter, and the activity level parameter to a predetermined temperature rate threshold, a predetermined heart rate threshold, a predetermined respiratory rate threshold, and a predetermined activity level threshold, respectively.

69. The method of any one of the previous Clauses, wherein the at least one controller is configured to determine at least one of a blood pH less than 7.2, a serum lactate greater than 2 mmol/L, a serum lactate greater than 4 mmol/L, a blood procalcitonin level above 2 ng/mL, and a low central venous pressure less than 2 mm/Hg.

70. The method of any one of the previous Clauses, wherein the sensing element is configured to obtain at least some of the physiological measurements continuously.

71. The method of any one of the previous Clauses, wherein the sensing element is configured to obtain at least some of the physiological measurements periodically.

72. The method of any one of the previous Clauses, wherein the at least one controller is configured to obtain at least some of the physiological measurements continuously.

73. The method of any one of the previous Clauses, wherein the at least one controller is configured to obtain at least some of the physiological measurements periodically.

74. The method of any one of the previous Clauses, wherein the at least one controller is configured to determine the at least one physiological parameter continuously.

75. The method of any one of the previous Clauses, wherein the at least one controller is configured to determine the at least one physiological parameter periodically.

76. The method of any one of the previous Clauses, wherein:
the sensing element is configured to measure temperature;
the at least one physiological parameter includes a temperature parameter; and
the at least one controller is configured to:
compare the temperature parameter to a predetermined temperature threshold;
determine the temperature parameter is outside of the predetermined temperature threshold; and
based on the determination, indicate that the patient is septic.

77. The method of any one of the previous Clauses, wherein the temperature parameter is a body temperature of the patient, and wherein determining the temperature parameter is outside of the predetermined temperature threshold includes determining the body temperature is outside of 96-100° F.

78. The method of any one of the previous Clauses, wherein the temperature parameter is a body temperature of the patient, and wherein determining the temperature parameter is outside of the predetermined temperature threshold includes determining the body temperature is outside of 96-100° F. for a predetermined amount of time.

79. The method of any one of the previous Clauses, wherein the temperature parameter is a body temperature of the patient, and wherein determining the temperature parameter is outside of the predetermined temperature threshold includes determining the body temperature is greater than 100° F.

80. The method of any one of the previous Clauses, wherein the temperature parameter is a body temperature of the patient, and wherein determining the temperature parameter is outside of the predetermined temperature threshold includes determining the body temperature is greater than 100° F. for a predetermined amount of time.

81. The method of any one of the previous Clauses, wherein the temperature parameter is a body temperature of the patient, and wherein determining the temperature parameter is outside of the predetermined temperature threshold includes determining the body temperature is less than 96° F.

82. The method of any one of the previous Clauses, wherein the temperature parameter is a body temperature of the patient, and wherein determining the temperature parameter is outside of the predetermined temperature threshold includes determining the body temperature is less than 96° F. for a predetermined amount of time.

83. The method of any one of the previous Clauses, wherein the temperature parameter is a change in a body temperature, and wherein determining the temperature parameter is outside of the predetermined temperature threshold includes determining at least a 2% change in body temperature from a baseline temperature.

84. The method of any one of the previous Clauses, wherein the temperature parameter is a change in a body temperature, and wherein determining the temperature parameter is outside of the predetermined temperature threshold includes determining at least a 2% change in body temperature from a baseline temperature over a predetermined amount of time.

85. The method of any one of the previous Clauses, wherein the at least one physiological parameter is a temperature parameter and a heart rate parameter.

86. The method of any one of the previous Clauses, wherein:
the sensing element comprises a first sensing element configured to measure temperature and a second sensing element configured to measure heart rate;
the at least one physiological parameter includes a temperature parameter and a heart rate parameter; and
the at least one controller is configured to:
compare the temperature parameter to a predetermined temperature threshold;
compare the heart rate parameter to a predetermined heart rate threshold;
determine the temperature parameter is outside of the predetermined temperature threshold;
determine the heart rate parameter is outside of the predetermined heart rate threshold; and
based on the determinations that the temperature parameter and the heart rate parameter are outside of the predetermined temperature threshold and the predetermined heart rate threshold, respectively, indicate that the patient is septic.

87. The method of any one of the previous Clauses, wherein the second sensing element comprises a pulse oximeter.

88. The method of any one of the previous Clauses, wherein the heart rate parameter is a heart rate of the patient, and wherein determining the heart rate parameter is outside of the predetermined threshold includes determining the heart rate of the patient is greater than 90 beats per minute.

89. The method of any one of the previous Clauses, wherein the heart rate parameter is a heart rate of the patient, and wherein determining the heart rate parameter is outside of the predetermined threshold includes determining the heart rate of the patient is greater than 90 beats per minute for a predetermined amount of time.

90. The method of any one of the previous Clauses, wherein the heart rate parameter is a change in a heart rate of the patient, and wherein determining the heart rate parameter is outside of the predetermined threshold includes determining the heart rate of the patient increases at least 10% from a reference heart rate of the patient.

91. The method of any one of the previous Clauses, wherein the heart rate parameter is a change in the patient's heart rate, and wherein determining the heart rate parameter is outside of the predetermined threshold includes determining the heart rate of the patient increases at least 10% from a reference heart rate of the patient over a predetermined amount of time.

92. The method of any one of the previous Clauses, wherein the at least one physiological parameter is a temperature parameter and a respiratory rate parameter.

93. The method of any one of the previous Clauses, wherein:
the sensing element comprises a first sensing element configured to measure temperature and a second sensing element configured to measure respiratory rate;
the at least one physiological parameter includes a temperature parameter and a respiratory rate parameter; and the at least one controller is configured to:
compare the temperature parameter to a predetermined temperature threshold;
compare the respiratory rate parameter to a predetermined respiratory rate threshold;
determine the temperature parameter is outside of the predetermined temperature threshold;
determine the respiratory rate parameter is outside of the predetermined respiratory rate threshold; and
based on the determinations that the temperature parameter and the respiratory rate parameter are outside of the predetermined temperature threshold and the predetermined respiratory rate threshold, respectively, indicate that the patient is septic.

94. The method of any one of the previous Clauses, wherein the respiratory rate parameter is a respiratory rate of the patient, and wherein determining the respiratory rate parameter is outside of the predetermined threshold includes determining the respiratory rate of the patient is greater than 15 breaths per minute.

95. The method of any one of the previous Clauses, wherein the respiratory rate parameter is a respiratory rate of the patient, and wherein determining the respiratory rate parameter is outside of the predetermined threshold includes determining the respiratory rate of the patient is greater than 15 breaths per minute for a predetermined amount of time.

96. The method of any one of the previous Clauses, wherein the at least one physiological parameter is a temperature parameter and an activity level parameter.

97. A system for monitoring the health of a patient, the system comprising:
a catheter configured to be implanted partially within a human patient, the catheter having a lumen;
a sensor coupled to the catheter and configured to obtain physiologic measurements; and
at least one controller configured to be communicatively coupled to the sensor, wherein the at least one controller is further configured to:
   obtain the physiological measurements via the sensor while the catheter is implanted within the patient;
   determine at least one physiological parameter based on the physiological measurements;
   compare the at least one physiological parameter to a predetermined threshold; and
   based on the comparison, provide an indication of the patient's health.

98. The system of Clause 97, wherein the sensor comprises at least one of a temperature sensor, a heart rate sensor, a respiratory rate sensor, a movement sensor, a pressure sensor, an electrical signal sensor, and an electro-optical sensor.

99. The system of any one of the previous Clauses, wherein the at least one controller is configured to provide an indication that the patient is clinically improving or decompensating based on the comparison.

100. The system of any one of the previous Clauses, wherein the at least one physiological parameter is at least one of a temperature parameter, a heart rate parameter, a respiratory rate parameter, and an activity level parameter.

101. The system of any one of the previous Clauses, wherein the at least one physiological parameter is at least two of a temperature parameter, a heart rate parameter, a respiratory rate parameter, and an activity level parameter.

102. The system of any one of the previous Clauses, wherein:
the at least one physiological parameter comprises at least two of a temperature parameter, a heart rate parameter, a respiratory rate parameter, and an activity level parameter;
comparing the at least two physiological parameters to the predetermined threshold includes comparing each of the at least two physiological parameters to a corresponding predetermined threshold.

103. The system of any one of the previous Clauses, wherein:
the at least one physiological parameter comprises at least three of a temperature parameter, a heart rate parameter, a respiratory rate parameter, and an activity level parameter;
comparing the at least three physiological parameters to the predetermined threshold includes comparing each of the at least three physiological parameters to a corresponding predetermined threshold.

104. The system of any one of the previous Clauses, wherein:
the at least one physiological parameter comprises a temperature parameter, a heart rate parameter, a respiratory rate parameter, and an activity level parameter;
comparing the at least one physiological parameter to the predetermined threshold includes comparing the temperature parameter, the heart rate parameter, the respiratory rate parameter, and the activity level parameter to a predetermined temperature rate threshold, a predetermined heart rate threshold, a predetermined respiratory rate threshold, and a predetermined activity level threshold, respectively.

105. The system of any one of the previous Clauses, wherein the at least one controller is configured to determine at least one of a blood pH less than 7.2, a serum lactate greater than 2 mmol/L, a serum lactate greater than 4 mmol/L, a blood procalcitonin level above 2 ng/mL, and a low central venous pressure less than 2 mm/Hg.

106. The system of any one of the previous Clauses, wherein the sensor is configured to obtain at least some of the physiological measurements continuously.

107. The system of any one of the previous Clauses, wherein the sensor is configured to obtain at least some of the physiological measurements periodically.

108. The system of any one of the previous Clauses, wherein the at least one controller is configured to obtain at least some of the physiological measurements continuously.

109. The system of any one of the previous Clauses, wherein the at least one controller is configured to obtain at least some of the physiological measurements periodically.

110. The system of any one of the previous Clauses, wherein the at least one controller is configured to determine the at least one physiological parameter continuously.

111. The system of any one of the previous Clauses, wherein the at least one controller is configured to determine the at least one physiological parameter periodically.

112. The system of any one of the previous Clauses, wherein:
the sensor is configured to measure temperature;
the at least one physiological parameter includes a temperature parameter; and
the at least one controller is configured to:
   compare the temperature parameter to a predetermined temperature threshold;
   determine the temperature parameter is outside of the predetermined temperature threshold; and
   based on the determination, indicate that the patient is ill.

113. The system of any one of the previous Clauses, wherein the temperature parameter is a body temperature of the patient, and wherein determining the temperature parameter is outside of the predetermined temperature threshold includes determining the body temperature is outside of 96-100° F.

114. The system of any one of the previous Clauses, wherein the temperature parameter is a body temperature of the patient, and wherein determining the temperature parameter is outside of the predetermined temperature threshold includes determining the body temperature is outside of 96-100° F. for a predetermined amount of time.

115. The system of any one of the previous Clauses, wherein the temperature parameter is a body temperature of the patient, and wherein determining the temperature parameter is outside of the predetermined temperature threshold includes determining the body temperature is greater than 100° F.

116. The system of any one of the previous Clauses, wherein the temperature parameter is a body temperature of the patient, and wherein determining the temperature parameter is outside of the predetermined temperature threshold includes determining the body temperature is greater than 100° F. for a predetermined amount of time.

117. The system of any one of the previous Clauses, wherein the temperature parameter is a body temperature of the patient, and wherein determining the temperature parameter is outside of the predetermined temperature threshold includes determining the body temperature is less than 96° F.

118. The system of any one of the previous Clauses, wherein the temperature parameter is a body temperature of the patient, and wherein determining the temperature parameter is outside of the predetermined temperature threshold includes determining the body temperature is less than 96° F. for a predetermined amount of time.

119. The system of any one of the previous Clauses, wherein the temperature parameter is a change in a body temperature, and wherein determining the temperature parameter is outside of the predetermined temperature threshold includes determining at least a 2% change in body temperature from a baseline temperature.

120. The system of any one of the previous Clauses, wherein the temperature parameter is a change in a body temperature, and wherein determining the temperature parameter is outside of the predetermined temperature threshold includes determining at least a 2% change in body temperature from a baseline temperature over a predetermined amount of time.

121. The system of any one of the previous Clauses, wherein the at least one physiological parameter is a temperature parameter and a heart rate parameter.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic representation of a monitoring system in accordance with embodiments of the present technology.

FIG. 2A is a top view of a junction configured to sense one or more parameters in accordance with embodiments of the present technology.

FIG. 2B is a cross-sectional view of the junction shown in FIG. 2A.

FIG. 3A is a vascular access assembly in accordance with embodiments of the present technology.

FIG. 3B is an enlarged, cross-sectional view of a portion of the vascular access assembly shown in FIG. 3A.

FIG. 4 is a schematic representation of a monitoring system in accordance with embodiments of the present technology.

FIGS. 5 and 6 are different views of a vascular monitoring device in accordance with several embodiments of the present technology.

FIGS. 7 and 8 show vascular monitoring devices of the present technology installed in and/or on a patient.

FIG. 9 is an isolated view of an external component of the vascular monitoring devices of the present technology.

FIG. 10 is an isolated view of an electronics component of the vascular monitoring devices of the present technology.

### DETAILED DESCRIPTION

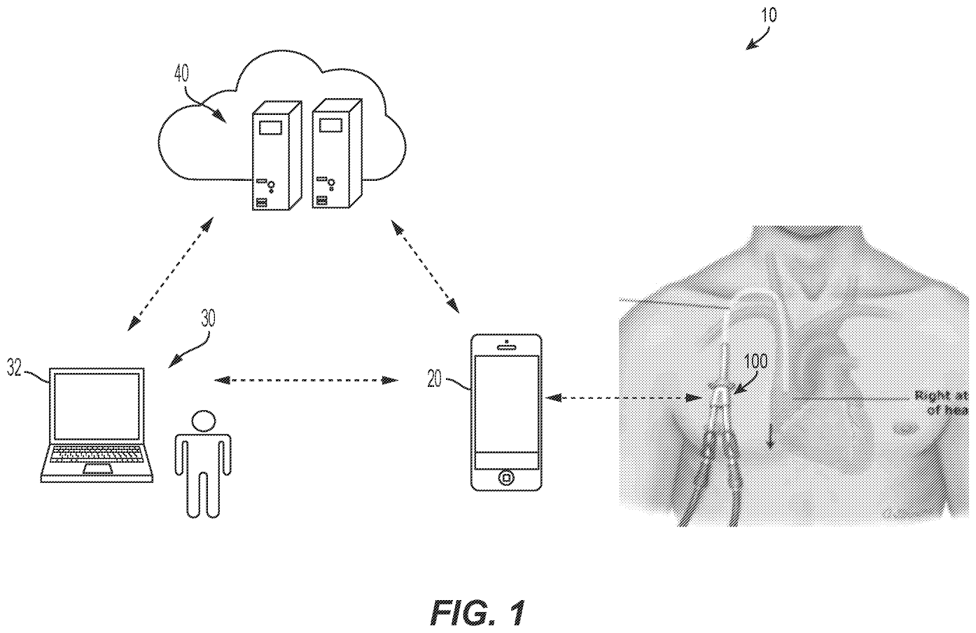

The present technology is directed to devices configured to provide or enable access to a blood vessel or other internal portion of a body of a patient. In particular, the present technology is related to vascular access devices comprising one or more sensors configured monitor one or more physiological parameters of the patient. Specific details of several embodiments of the technology are described below with reference to FIGS. 1-10.

### I. Overview

FIG. 1 is a schematic representation of a treatment system 10 for monitoring the health of a patient via a vascular access assembly 100 (or “VAA 100”) having one or more sensors in accordance with the present technology. The sensors may be configured to obtain physiological measurements that are used by the system 10 to determine one or more physiological parameters indicative of the patient's health. Additionally or alternatively, the sensors may be configured to obtain one or more local parameters indicative of the operation of one or more components of the VAA 100 and/or the local environment. In some embodiments, the system 10 may detect a medical condition (such as sepsis) or associated symptom(s) based on the physiological parameter(s) and provide an indication of the detected symptom or condition to the patient, caregiver, and/or medical care team.

The VAA 100 may be any device or system configured to provide access to a patient's blood vessel from an extracorporeal location. For example, the VAA 100 may be a central venous catheter (“CVC”), an arterial line, a midline catheter, a peripheral intravenous line, and other tunneled and non-tunneled catheters. In some embodiments, the VAA 100 may be a PICC (peripherally inserted central catheter) line. In any case, the VAA 100 may be configured to administer pain medication, administer antibiotics, sample blood, perform a blood transfusion, administer chemotherapy, hydration, total parenteral nutrition, hemodialysis, apheresis, and other long term fluid administration applications.

As shown schematically in FIG. 1, one or more components of the VAA 100 may be configured to communicate wirelessly with a local computing device 20, which can be, for example, a smart device (e.g., a smartphone, a tablet, or other handheld device having a processor and memory), a special-purpose interrogation device, or other suitable device. Communication between the VAA 100 and the local computing device 20 can be mediated by, for example, near-field communication (NFC), infrared wireless, Bluetooth, ZigBee, Wi-Fi, inductive coupling, capacitive coupling, or any other suitable wireless communication link. The VAA 100 may transmit data including, for example, physiological measurements obtained via the sensor, patient medical records, device performance metrics (e.g., battery level, error logs, etc.), or any other such data stored by the VAA 100. In some embodiments, the transmitted data is encrypted or otherwise obfuscated to maintain security during transmission to the local computing device 20. The local computing device 20 may also provide instructions to the VAA 100, for example to obtain certain physiological measurements via the sensor, to emit a localization signal, or to perform other functions. In some embodiments, the local computing device 20 may be configured to wirelessly recharge a battery of the VAA 100, for example via inductive charging.

The system 10 may further include first remote computing device(s) 40 (or server(s)), and the local computing device 20 may in turn be in communication with first remote computing device(s) 40 over a wired or wireless communications link (e.g., the Internet, public and private intranet, a local or extended Wi-Fi network, cell towers, the plain old telephone system (POTS), etc.). The first remote computing device(s) 40 may include one or more own processor(s) and memory. The memory may be a tangible, non-transitory computer-readable medium configured to store instructions executable by the processor(s). The memory may also be configured to function as a remote database, i.e., the memory may be configured to permanently or temporarily store data received from the local computing device 20 (such as one or more physiological measurements or parameters and/or other patient information).

In some embodiments, the first remote computing device(s) 40 can additionally or alternatively include, for example, server computers associated with a hospital, a medical provider, medical records database, insurance company, or other entity charged with securely storing patient data and/or device data. At a remote location 30 (e.g., a hospital, clinic, insurance office, medical records database, operator's home, etc.), an operator may access the data via a second remote computing device 32, which can be, for example a personal computer, smart device (e.g., a smartphone, a tablet, or other handheld device having a processor and memory), or other suitable device. The operator may access the data, for example, via a web-based application. In some embodiments, the obfuscated data provided by the VAA 100 can be de-obfuscated (e.g., unencrypted) at the remote location 30.

In some embodiments, the VAA 100 may communicate with remote computing devices 32 and/or 40 without the intermediation of the local computing device 20. For example, the VAA 100 may be connected via Wi-Fi or other wireless communications link to a network such as the Internet. In other embodiments, the VAA 100 may be in communication only with the local computing device 20, which in turn is in communication with remote computing devices 32 and/or 40.

### II. Selected Embodiments of Junctions

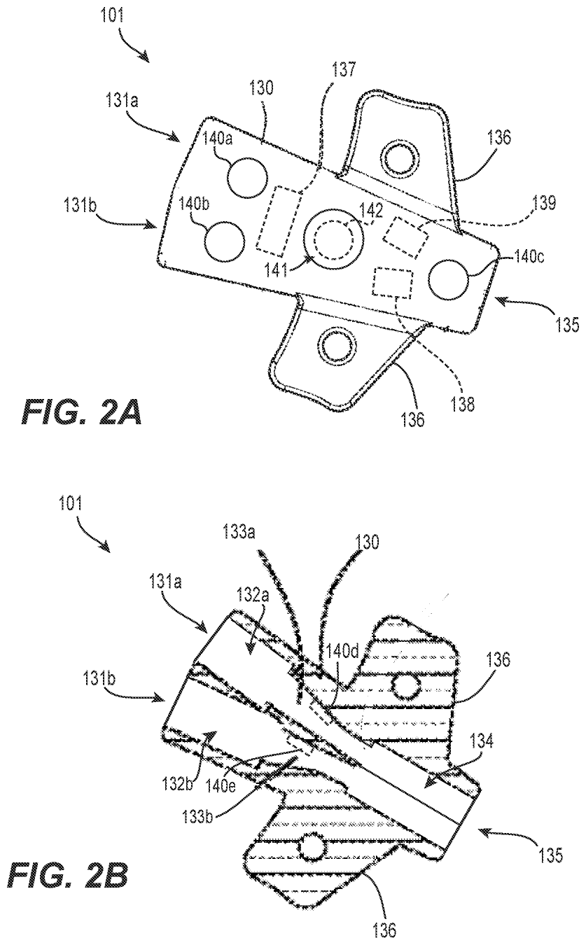

FIG. 2A is a top view of a catheter junction 101 configured to sense one or more physiological or operational parameters in accordance with the present technology. FIG. 2B shows the junction 101 in cross section. The junction 101 is configured to provide fluid communication between one or more extracorporeally-positioned fluid sources (or receptacles) and one or more catheters implanted in a patient. The junction 101, for example, may be configured for use with a VAA, such as any of the VAA's disclosed herein (e.g., VAA 100, VAA 200, etc.). In use, all or a portion of the junction 101 may be extracorporeally positioned so that the junction 101 can be accessed by the patient and/or a caregiver. Most commonly, when in use, the junction 101 may be positioned at an upper region of the patient's chest or at a patient's upper or lower arm.

In the depicted embodiment, the junction 101 is configured to fluidly couple up to two distinct fluid sources with a single distal catheter (which could have any number of lumens). As such, the junction 101 includes first and second inflow regions that merge into a single outflow region. The first inflow region extends distally from a first opening 131a in the body of the junction 101 to a first receiving region 132a, then to a first connecting region 133a that feeds into the outflow region. The first receiving region 132a is configured to couple to a fluid source detachably or permanently. For example, the first receiving region 132a may be configured to receive an end portion of a catheter tube (such as an extension tube), the distal end portion of a syringe, and others. The second inflow region extends distally from a second opening 131b in the body of the junction 101 to a second receiving region 132b, then to a second connecting region 133b that feeds into the outflow region. The second connecting region 133b is separate and distinct from the first connecting region 133a. Similar to the first receiving region 132a, the second receiving region 132b is configured to couple to a fluid source detachably or permanently. The outflow region may extend proximally from a third opening 135 in the body of the junction 101 to a receiving region 134. The receiving region 134 may be configured to couple to a catheter detachably or permanently. For example, the receiving region 134 may be configured to receive an end portion of a catheter therein. In some embodiments, the receiving region 134 may be configured to receive an end portion of multiple catheters therein.

In some embodiments, the junction 101 may be heat molded to its desired shape. For instance, the junction 101 may be formed of a unitary body within which the inflow and outflow regions have been formed. In some embodiments, the junction 101 may comprise two or more pre-molded pieces that fit together to form the junction 101. In any case, the junction 101 may taper towards the outflow region, and may include one or more fixation wings 136. In some embodiments, the junction 101 does not have a tapered portion and/or does not include any fixation wings 136. The junction 101 can have other suitable shapes, sizes, and configurations.

It will be appreciated that the junctions of the present technology are configured to receive more or fewer than two fluid sources (e.g., one fluid source, three fluid sources, four fluid sources, etc.). For example, the junctions of the present technology may include one, three, four, etc. inflow regions and/or connecting regions. Likewise, the junctions of the present technology may be configured to receive more than one distal catheter. For example, the junctions of the present technology may include two, three, four, or more distinct outflow regions.

The junction 101 may include one or more sensors configured to obtain physiological or operational measurements, as well as a controller 137 communicatively coupled to the one or more sensors, an antenna or data communications unit 138, and a battery 139. In some embodiments, the junction 101 does not include at least one of the controller 137, the antenna 138, and the battery 139. The controller 137 may include one or more processors, software components, and memory (not shown). In some examples, the one or more processors include one or more computing components configured to process the measurements received from the sensor(s) according to instructions stored in the memory. The memory may be a tangible, non-transitory computer-readable medium configured to store instructions executable by the one or more processors. For instance, the memory may be data storage that can be loaded with one or more of the software components executable by the one or more processors to achieve certain functions. In some examples, the functions may involve causing the sensor(s) to obtain measurements, which may include physiological data from the patient. In another example, the functions may involve processing the physiological data to determine one or more physiological parameters and/or provide an indication to the patient and/or clinician of one or more symptoms or medical conditions associated with the determined physiological parameters or symptoms.

The data communications unit 138 may be configured to securely transmit data between the junction 101 and external computing devices (e.g., local computing device 20, remote computing devices 32 and 40, etc.). In some embodiments, the communications unit may include a wireless communication module, such as a Bluetooth Low Energy chip or similar module configured to enable short-range or long-range wireless communication between the junction 101 and one or more remote computing devices. The controller 137 can also include a wireless charging unit (such as a coil) configured to recharge the battery 139 of the junction 101 when in the presence of an interrogation device (e.g., local device 20 or another suitable device). In some embodiments, individual electronics components may include a plurality of individual elements mounted to one or both sides of a printed circuit board (PCB). According to some aspects of the technology, one or more of the electronics components, such as the controller 137, the data communications unit 138, and/or the battery 139 may be included as a separate component that is coupled to the junction 101 and/or another component of the associated VAA. In some embodiments, one or more of the electronics components may be incorporated into one or both of the fixations wings 136.

Referring still to FIGS. 2A and 2B, the junction 101 may include one or more sensors disposed at an exterior and/or interior location of the junction 101. As used herein, the term “sensor” may refer to a single sensor or a plurality of discrete, separate sensors. The sensors and/or controller 137 may be configured to measure and/or calculate temperature, moisture, tumescence, impedance, pressure, heart rate, respiratory rate, and other parameters. In some embodiments, the sensors and/or controller 137 may identify, monitor, and communicate patient information by electromagnetic, acoustic, motion, optical, thermal, or biochemical sensors or means. Any of the sensors may include, for example, one or more temperature sensors (e.g., one or more thermocouples, one or more digital temperature sensors, one or more thermistors or other type of resistance temperature detector, etc.), one or more impedance sensors (e.g., one or more electrodes), one or more pressure sensors, one or more optical sensors (e.g., one or more pulse oximeters), one or more flow sensors (e.g., a Doppler velocity sensor, an ultrasonic flow meter, etc.), one or more ultrasonic sensors, one or more chemical sensors, one or more movement sensors (e.g., one or more accelerometers), one or more pH sensors, an electrocardiogram (“ECG” or “EKG”) unit, one or more electrochemical sensors, one or more hemodynamic sensors, and/or other suitable sensing devices.

Any of the sensors may comprise one or more electromagnetic sensors configured to measure and/or detect, for example, impedance, voltage, current, or magnetic field sensing capability with a wire, wires, wire bundle, magnetic node, and/or array of nodes. The sensors may comprise one or more acoustic sensors configured to measure and/or detect, for example, sound frequency, within human auditory range or below or above frequencies of human auditory range, beat or pulse pattern, tonal pitch melody, and/or song. The sensors may comprise one or more motion sensors configured to measure and/or detect, for example, vibration, movement pulse, pattern or rhythm of movement, intensity of movement, and/or speed of movement. Motion communication may occur by a recognizable response to a signal. This response may be by vibration, pulse, movement pattern, direction, acceleration, or rate of movement. Motion communication may also be by lack of response, in which case a physical signal, vibration, or bump to the environment yields a motion response in the surrounding tissue that can be distinguished from the motion response of the sensor. Motion communication may also be by characteristic input signal and responding resonance.

The sensor may comprise one or more optical sensors which may include, for example, illuminating light wavelength, light intensity, on/off light pulse frequency, on/off light pulse pattern, passive glow or active glow when illuminated with special light such as UV or “black light”, or display of recognizable shapes or characters. It also includes characterization by spectroscopy, interferometry, response to infrared illumination, and/or optical coherence tomography. The sensor(s) may comprise one or more thermal sensors configured to measure and/or detect, for example, junction 101 temperature relative to surrounding environment, the temperature of the junction 101 (or portion thereof), the temperature of the environment surrounding the junction 101 and/or sensor, or differential rate of the device temperature change relative to surroundings when the device environment is heated or cooled by external means. The sensor(s) may comprise one or more biochemical devices which may include, for example, the use of a catheter, a tubule, wicking paper, or wicking fiber to enable micro-fluidic transport of bodily fluid for sensing of protein, RNA, DNA, antigen, and/or virus with a micro-array chip.

In some aspects of the technology, the controller 137 and/or sensor(s) may be configured to detect and/or measure the concentration of blood constituents, such as sodium, potassium, chloride, bicarbonate, creatinine, blood urea nitrogen, calcium, magnesium, and phosphorus. The system 10 and/or the sensors may be configured to evaluate liver function (e.g., by evaluation and/or detection of AST, ALT, alkaline phosphatase, gamma glutamyl transferase, troponin, etc.), heart function (e.g., by evaluation and/or detection of troponin), coagulation (e.g., via determination of prothrombin time (PT), partial thromboplastin time (PTT), and international normalized ratio (INR)), and/or blood counts (e.g., hemoglobin or hematocrit, white blood cell levels with differential, and platelets). In some embodiments, the system 10 and/or sensor(s) may be configured to detect and/or measure circulating tumor cells, circulating tumor DNA, circulating RNA, multigene sequencing of germ line or tumor DNA, markers of inflammation such as cytokines, C reactive protein, erythrocyte sedimentation rate, tumor markers (PSA, beta-HCG, AFP, LDH, CA 125, CA 19-9, CEA, etc.), and others.

As previously mentioned, the junction 101, associated VAA, and/or system 10 may determine one or more physiological parameters based on the physiological measurements and/or one or more other physiological parameter(s). For example, the junction 101, associated VAA, and/or system 10 may be configured to determine physiological parameters such as heart rate, temperature, blood pressure (e.g., systolic blood pressure, diastolic blood pressure, mean blood pressure), blood flow rate, blood velocity, pulse wave speed, volumetric flow rate, reflected pressure wave amplitude, augmentation index, flow reserve, resistance reserve, resistive index, capacitance reserve, hematocrit, heart rhythm, electrocardiogram (ECG) tracings, body fat percentage, activity level, body movement, falls, gait analysis, seizure activity, blood glucose levels, drug/medication levels, blood gas constituents and blood gas levels (e.g., oxygen, carbon dioxide, etc.), lactate levels, hormone levels (such as cortisol, thyroid hormone (T4, T3, free T4, free T3), TSH, ACTH, parathyroid hormone), and/or any correlates and/or derivatives of the foregoing measurements and parameters (e.g., raw data values, including voltages and/or other directly measured values). In some embodiments, one or more of the physiological measurements can be utilized or characterized as a physiological parameter without any additional processing by the junction 101, associated VAA, and/or system 10.

The junction 101, associated VAA, and/or system 10 may also determine and/or monitor derivatives of any of the foregoing physiological parameters (also referred to herein as “physiological parameters”), such as a rate of change of a particular parameter, a change in a particular parameter over a particular time frame, etc. As but a few examples, the junction 101, associated VAA, and/or system 10 may be configured to determine as temperature over a specified time, a maximum temperature, a maximum average temperature, a minimum temperature, a temperature at a predetermined or calculated time relative to a predetermined or calculated temperature, an average temperature over a specified time, a maximum blood flow, a minimum blood flow, a blood flow at a predetermined or calculated time relative to a predetermined or calculated blood flow, an average blood flow over time, a maximum impedance, a minimum impedance, an impedance at a predetermined or calculated time relative to a predetermined or calculated impedance, a change in impedance over a specified time, a change in impedance relative to a change in temperature over a specified time, a change in heart rate over time, a change in respiratory rate over time, activity level over a specified time and/or at a specified time of day, and other suitable derivatives.

Measurements may be obtained continuously or periodically at one or more predetermined times, ranges of times, calculated times, and/or times when or relative to when a measured event occurs. Likewise, physiological parameters may be determined continuously or periodically at one or more predetermined times, ranges of times, calculated times, and/or times when or relative to when a measured event occurs.

Referring still to FIGS. 2A and 2B, in some embodiments, one or more of the sensors may be built into the body of the junction 101 such that only a portion of the respective sensor is exposed to the local physiological environment (e.g., the patient's skin) when the junction 101 is in use. For instance, the sensor may comprise one or more electrodes having an external portion positioned at an exterior surface of the junction body and an internal portion positioned within the junction body and wired to another sensor, a controller (described below), and/or another component of the VAA (such as an extension tube or distal catheter, as described below).

In the example depicted at FIG. 2A, the junction 101 includes first, second, and third electrodes 140a, 140b, 140c, all disposed at the same side of the junction 101 and configured to be in contact with the patient's skin when the junction 101 is in use and positioned on the patient. Each of the sensors 140a, 140b, 140c may be positioned near a different inflow or outflow region. In these and other embodiments, the junction 101 may include more or fewer than three sensors, and the sensors be positioned anywhere along the exterior surface of the junction 101. For example, in some embodiments both sides of the junction 101 may include one or more exterior sensors, and in some embodiments one or both of the fixation wings 136 may include one or more sensors.

It may be beneficial to include sensors at an exterior portion of the junction 101 to obtain data indicative of patient health and/or device operation. For example, the inclusion of one or more electrodes configured to measure impedance can be used to confirm contact between the electrodes and the patient's skin. The ability confirm contact between the electrodes and the patient's skin can be used to validate measurements obtained from other sensors, such as temperature, moisture, heart rate, skin color, etc.

In some embodiments the sensor(s) may be completely contained within the body of the junction 101. For example, the sensor(s) may comprise one or more optical sensors 142 (such as one or more pulse oximeters) enclosed by the junction body. The junction body may include a window 141 substantially aligned with the optical sensor 142 and through which light emitted from the optical sensor may pass to an external location, and back through which light reflected from the external location may pass for detection by a photodiode of the optical sensor. In such embodiments the window may be, for example, a sapphire window that is brazed into place within an exterior region of the junction body.

According to several aspects of the technology, the junction 101 may include one or more sensors disposed at an interior location. For example, as shown in FIG. 2B, the junction may include a sensor 140d positioned within the first connecting region 133a and a sensor 140e positioned within the second connecting region 133b. As such, the sensors 140d, 140e will be directly exposed to any fluid flowing through the respective connecting region 133 (i.e., between the respective inflow receiving region and the outflow receiving region). In some embodiments, all of the connecting regions 133 include a sensor. In some embodiments, less than all of the connecting regions 133 include a sensor (including none of the connecting regions 133). According to some aspects of the technology, the junction 101 may include one or more sensors at other interior regions, such as at the first and/or second receiving region 132a, 132b and/or the outflow receiving region 134. It may be beneficial to position one or more sensors within a connecting region to measure the pressure of the flow, the chemical composition of the fluid flowing through the connecting region, the temperature of the fluid flowing through the connecting region, and others.

In some embodiments, one or more of the sensors may include a separate controller (not shown) that comprises one or more processors and/or software components. In such embodiments, the sensor(s) may process at least some of the physiological measurements to determine one or more physiological parameters, and then transmit those physiological parameters to the controller 137 of the junction 101 (with or without the underlying physiological data). In some examples, the sensor(s) may only partially process at least some of the physiological measurements before transmitting the data to the controller 137. In such embodiments, the controller 137 may further process the received physiological data to determine one or more physiological parameters. The local computing device 20 and/or the remote computing devices 32, 40 may also process some or all of the physiological measurements obtained by the sensor(s) and/or physiological parameters determined by the sensor(s) and/or the controller 137.

### III. Selected Embodiments of Vascular Access Assemblies

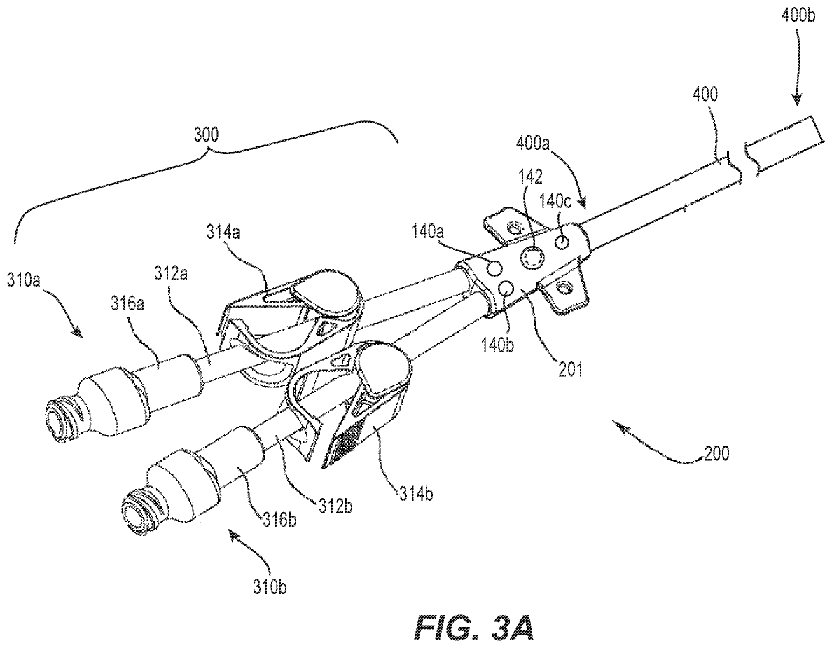

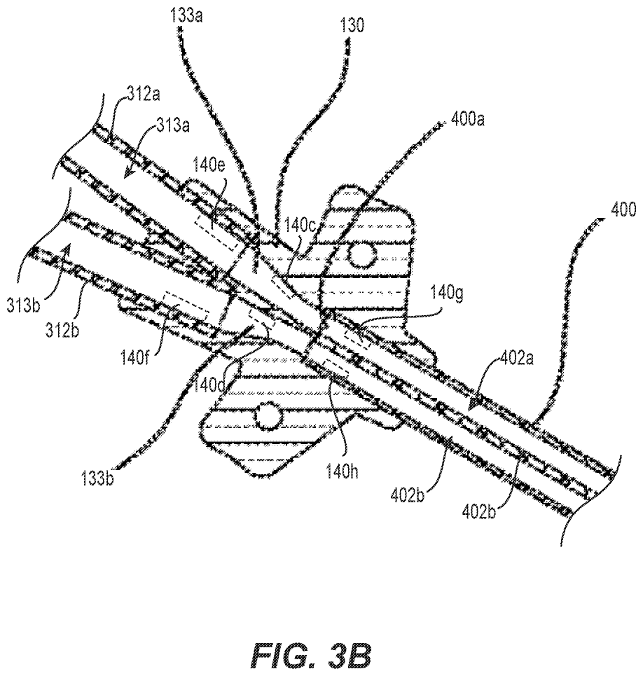

FIG. 3A shows a VAA 200 configured in accordance with the present technology, and FIG. 3B shows an enlarged cross-sectional view of the region of the VAA 200 at the junction 101. The VAA 200 may have features that are generally similar to the VAA 100 described above. For example, the VAA 200 may be configured to be used within the system 10. In general, the VAA 200 may be any device or system configured to provide access to a patient's blood vessel from an extracorporeal location. For example, the VAA 200 may be a CVC, an arterial line, a midline catheter, a peripheral intravenous line, and other tunneled and non-tunneled catheters. In some embodiments, the VAA 200 may be a PICC line. In any case, the VAA 200 may be configured to administer pain medication, administer antibiotics, sample blood, perform a blood transfusion, administer chemotherapy, hydration, total parenteral nutrition, hemodialysis, and other long term fluid administration applications.

As shown, the VAA 200 includes a proximal assembly 300, a distal catheter 400, and a junction 101 disposed between the proximal assembly 300 and the distal catheter 400. The proximal assembly 300 may include first and second legs 310a and 310b (referred to collectively as “legs 310”), each comprising an extension tube 312a, 312b, a flow control 314a, 314b, and a connector 316a, 316b. The extension tubes 312a, 312b define corresponding lumens 313a, 313b (FIG. 3B) extending therethrough. The flow controls 314a, 314b may comprise one or more valves or clamps coupled to a respective extension tube 312a, 312b and configured to control the flow of fluid within the respective extension tube 312a, 312b. The connectors 316a, 316b may be disposed at the proximal end portions of the respective extension tubes 312a, 312b and are configured to be coupled to a fluid source, such as a syringe or IV bag. In some embodiments, the connectors 316a, 316b are configured to be coupled to a fluid repository, such as a blood collection bag. In some embodiments, the proximal assembly 300 may comprise more or fewer legs 310.

The distal catheter 400 may comprise an elongated tubular shaft extending between a proximal end portion 400a and a distal end portion 400b. The distal catheter 400 may define first and second lumens 402a, 402b (FIG. 3B) extending therethrough separated by a longitudinally-extending septum 404. In some embodiments, the distal catheter 400 may have more or fewer lumens. For example, the distal catheter 400 may be a single, double, triple, or quadruple lumen catheter. In use, all or a portion of the distal catheter 400 is configured to be implanted within the patient, for example at a subcutaneous or intravascular location.

The junction 201 can be generally similar to the junction 101 discussed above with reference to FIGS. 2A and 2B. As shown in the enlarged, cross-sectional view of FIG. 3B, a distal end portion of the first extension tube 312a is configured to be received within the first receiving region 132a of the first inflow region of the junction 101, and a distal end portion of the second extension tube 312b is configured to be received within the second receiving region 132b of the second inflow region of the junction 101. In some embodiments, a first and second sensor 140e, 140f may be disposed in the corresponding lumen of the first and second extension tubes 312a, 312b. In some embodiments, both or neither of the extension tubes 312a, 312b include a sensor.

A proximal end portion of the distal catheter 400 is configured to be received within the receiving region of the outflow region of the junction, as shown in FIG. 3B. In some embodiments, the distal catheter 400 may include one or more sensors disposed at or along it sidewalls and/or septum 404. In those embodiments where the distal catheter 400 includes multiple lumens (such as the present embodiment), the distal catheter 400 may include a first sensor 140g located in its first lumen 402a and a second sensor 140h located in its second lumen 402b. In some embodiments, all, some, or none of the distal catheter lumens include a sensor. In some embodiments, the sensors and/or wires may be coextruded in the catheter wall or constrained within one or more catheter lumens in fluid communication with the body, etc.

The sensors incorporated into the proximal assembly and/or distal catheter may be any of the sensors detailed herein.

### IV. Selected Embodiments of Arterial Pressure Monitoring Devices, Systems, and Methods

Critically ill patients require aggressive medical treatment and continuous monitoring, typically in the hospital setting. Numerous injuries and illnesses can lead to a critical condition including sepsis, cardiovascular disorders such as myocardial infarction and cerebrovascular accident, thromboembolic disorders, renal failure, liver failure, hemorrhage, and trauma. Critical patients often have underlying medical comorbidities which complicate their management. Within the hospital, critical patients are often located in the emergency department, intensive care unit, operating room, or so called “step-down units.” Critical care can also take place en route to the hospital in an ambulance, air transport unit, or other advanced life support capable transport vehicle.

Critical care requires continuous monitoring of patient status including vital signs. As such, critical patients will frequently undergo placement of an arterial pressure monitor (“arterial line” or “art-line”) which provides invasive arterial pressure data including systolic blood pressure, diastolic blood pressure, arterial waveform, pulse pressure, mean arterial pressure (MAP), and heart rate. Arterial line data is used to monitor for acute status changes and to inform treatment decisions. For example, critical patients may be on “pressor” medications (i.e. Levophed, Neo-Synephrine) that cause vasoconstriction and support blood pressure. These medications are administered as a continuous infusion or “drip,” and the rate of infusion is closely titrated based on the arterial pressure readings.

Current arterial monitoring devices require a physical connection to an external manometer. A flexible vascular catheter (such as an angiocath) is typically inserted into the radial artery. From there, the catheter is flushed with saline and connected via tubing (also filled with saline) to a manometer for measuring physical pressure within the system. This current system therefore requires a physical connection between the patient and a large external system, which comes with a host of potential complications and problems. Tangling of lines and tubes can lead to inaccurate data, or worse, inadvertent removal of the catheter from the artery requiring replacement. The physical connection also complicates patient transport for procedures and diagnostic testing, typically leading to disconnecting the arterial pressure monitor prior to moving the patient. During transport, the patient is no longer monitored. To address these challenges, the present technology comprises an easy to use, form fitting, and wireless invasive arterial pressure monitor for using in critical care.

The arterial pressure monitoring device described herein is designed to provide accurate, diagnostic, continuous arterial pressure monitoring for hospitalized or other critical patients. In addition to monitoring arterial pressure, the device is configured to monitor heart rate, cardiac rhythm, respiratory rate, oxygen saturation, and arterial blood gas parameters including partial pressure of oxygen, partial pressure of carbon dioxide, pH, and bicarbonate.

Despite its invasive nature, the arterial monitoring device of the present technology is designed for ease of use and patient comfort. Wireless connectivity eliminates the need for a connection to an external manometer and facilitates monitoring of patients at all times, including during transport. The entire device can be contained within an integrated unit that includes an invasive component placed within an artery connected to an external component that holds the invasive component in place and contains hardware for power supply, data acquisition, processing, and communication.

According to some aspects of the technology, the device described herein is configured to be secured to the patient's wrist. The device may include an external component configured to be electrically coupled to a biocompatible elongated conductor, such as a fiber optic connection and/or wire that is configured to be inserted into a blood vessel. For example, in some embodiments the elongated conductor is configured to be positioned within an artery, such as the radial (or ulnar) artery. In those embodiments including a wire, the wire may comprise nitinol, stainless steel, or another metal or metal alloy. The wire may be formed of a superelastic and/or resilient material, and in some embodiments may be biased towards a preset shape. The external component and/or elongated conductor can be configured to be electrically coupled to one or more sensors, such as, for example, a pressure sensor for continuous monitoring of arterial pressure. In some embodiments the external component includes one or more integrated sensors. In other embodiments, the external component does not include any integrated sensors. In any case, the device may include one or more sensors positioned on the elongated conductor. The sensor(s) may be positioned at any location along the length of the elongated conductor. In some embodiments the device includes one or more sensors at a distal portion of the elongated conductor. According to several embodiments, the device includes one or more sensor at a distal tip of the elongated conductor.

Other sensors include temperature sensors, such as a thermistor or temperature sensing chip, an oxygen saturation monitor, specialized biosensors for measuring body or blood chemistry parameters or hematologic values, and others. The external component and/or elongated conductor may additionally or alternatively include one or more electrodes for monitoring cardiac rate and rhythm.

In some embodiments the external component may comprise a wearable member, such as a patch, a wrist band, a leg band, an arm band, and others. The elongated conductor can be physically connected to the wearable member that fits tightly over the patient's extremity (and/or other body portion) and holds the intraarterial elongated conductor in position. The elongated conductor can be in physical and electrical communication with an electronics component carried by the wrist band. The electronics component can include wires and/or other electrical connectors for electrical signal and power conduction, a programmable micro-controller unit, memory, a power source such as a battery, sensor chips, and an antenna for wireless communication.

According to some embodiments, the vascular monitoring device includes a vascular component coupled to the external component. The vascular component, for example, can comprise a catheter, such as an arterial catheter. In some embodiments, the vascular component is an angiocatheter. The vascular component may be configured to be inserted into a blood vessel (such as an artery, including a radial artery). In some embodiments, the vascular component is filled with a fluid, such as saline. In these and other embodiments, the device includes a pressure sensor on or in the external component, and the vascular component is in fluid communication with the pressure sensor. The vascular component can include one or more elongated conductors, such as a wire and/or fiber optic connections, for additional sensor function as described above.

In some embodiments the device includes a receptacle, which can be integrated with the external component. According to several versions of these embodiments, the device includes a catheter (as described herein) configured to be fluidly coupled to the receptacle. The receptacle can comprise a chamber and/or a microfluidic channel(s) housed by the external component, and/or in some embodiments may be a chip containing a microfluidic array, where the external component is configured to receive the chip. In some embodiments, the external component includes a removable cartridge. According to some aspects of the technology, the receptacle can be a separate component configured to be fluidly coupled to the external component. For example, the device can include an external catheter and/or a separate chamber fluidly coupled to the external component and/or vascular catheter. In any case, blood, such as arterial blood, may be aspirated from the artery into the receptacle within the external component allowing for measurement of blood parameters such as arterial blood gasses (partial pressure of oxygen, partial pressure or carbon dioxide, bicarbonate, and pH), hematologic parameters such as hemoglobin, hematocrit, white blood cell count with differential, and platelets, blood chemistry parameters such as electrolyte levels, kidney function tests, liver function tests, tumor markers, D-Dimer, cardiac enzymes, lactate, prolactin, or other laboratory testing. Aspiration can take place through an automated suction and pumping system, or by a healthcare provider attaching a syringe or other device to apply negative pressure to the system in fluid communication with the arterial catheter.

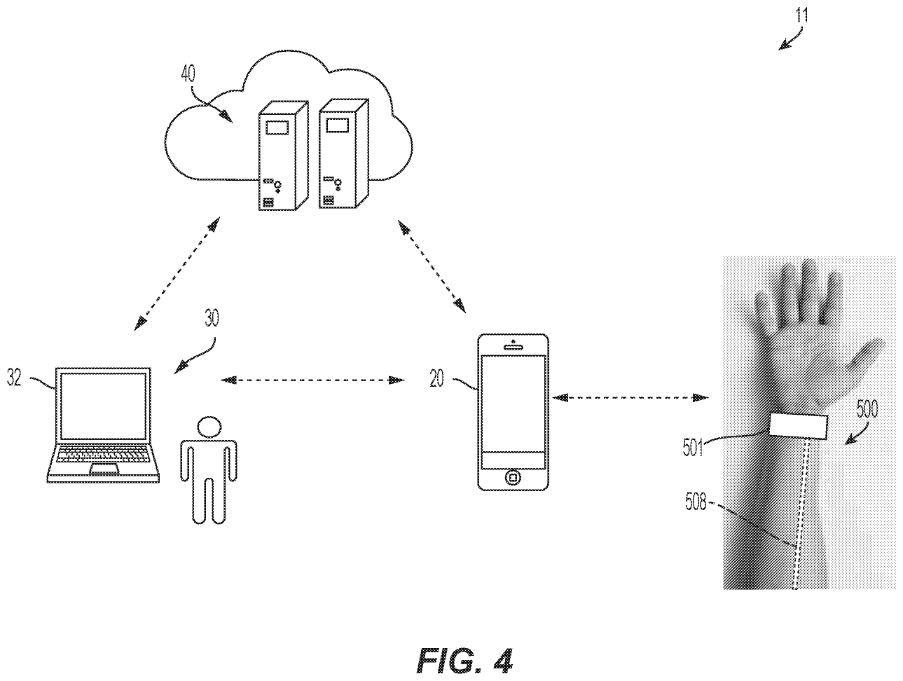

FIG. 4 is a schematic representation of an example treatment system 11 for monitoring the health of a patient via a VAA, such as a vascular monitoring device 500 (or “device 500”). In some embodiments, the device 500 is an arterial monitoring device. Several of the components of the system 11 can be substantially similar to the components of system 10.

In some embodiments, the device 500 may comprise one or more sensors and a vascular component 508 (such as an arterial catheter and/or elongated conductor) configured to obtain physiological measurements that are used by the system 11 to determine one or more physiological parameters indicative of the patient's health. In FIG. 4, the vascular component is shown positioned in the radial artery, although it will be appreciated that other insertion locations (such as within other blood vessels and/or other arteries) are possible. In any case, the sensors may be configured to obtain one or more local parameters indicative of the operation of one or more components of the device 500 and/or the local environment. In some embodiments, the system 11 may detect a medical condition (such as sepsis) or associated symptom(s) based on the physiological parameter(s) and provide an indication of the detected symptom or condition to the patient, caregiver, and/or medical care team.

As shown schematically in FIG. 4, one or more components of the device 500 may be configured to communicate wirelessly with a local computing device 20, which can be, for example, a smart device (e.g., a smartphone, a tablet, or other handheld device having a processor and memory), a special-purpose interrogation device, or other suitable device. Communication between the device 500 and the local computing device 20 can be mediated by, for example, near-field communication (NFC), infrared wireless, Bluetooth, ZigBee, Wi-Fi, inductive coupling, capacitive coupling, or any other suitable wireless communication link. The device 500 may transmit data including, for example, physiological measurements obtained via the sensor, patient medical records, device performance metrics (e.g., battery level, error logs, etc.), or any other such data stored by the device 500. In some embodiments, the transmitted data is encrypted or otherwise obfuscated to maintain security during transmission to the local computing device 20. The local computing device 20 may also provide instructions to the device 500, for example to obtain certain physiological measurements via the sensor, to emit a localization signal, or to perform other functions. In some embodiments, the local computing device 20 may be configured to wirelessly recharge a battery of the device 500, for example via inductive charging.

The system 11 may further include first remote computing device(s) 40 (or server(s)), and the local computing device 20 may in turn be in communication with first remote computing device(s) 40 over a wired or wireless communications link (e.g., the Internet, public and private intranet, a local or extended Wi-Fi network, cell towers, the plain old telephone system (POTS), etc.). The first remote computing device(s) 40 may include one or more own processor(s) and memory. The memory may be a tangible, non-transitory computer-readable medium configured to store instructions executable by the processor(s). The memory may also be configured to function as a remote database, i.e., the memory may be configured to permanently or temporarily store data received from the local computing device 20 (such as one or more physiological measurements or parameters and/or other patient information).

In some embodiments, the first remote computing device(s) 40 can additionally or alternatively include, for example, server computers associated with a hospital, a medical provider, medical records database, insurance company, or other entity charged with securely storing patient data and/or device data. At a remote location 30 (e.g., a hospital, clinic, insurance office, medical records database, operator's home, etc.), an operator may access the data via a second remote computing device 32, which can be, for example a personal computer, smart device (e.g., a smartphone, a tablet, or other handheld device having a processor and memory), or other suitable device. The operator may access the data, for example, via a web-based application. In some embodiments, the obfuscated data provided by the device 500 can be de-obfuscated (e.g., unencrypted) at the remote location 30.

In some embodiments, the device 500 may communicate with remote computing devices 32 and/or 40 without the intermediation of the local computing device 20. For example, the device 500 may be connected via Wi-Fi or other wireless communications link to a network such as the Internet. In other embodiments, the device 500 may be in communication only with the local computing device 20, which in turn is in communication with remote computing devices 32 and/or 40.

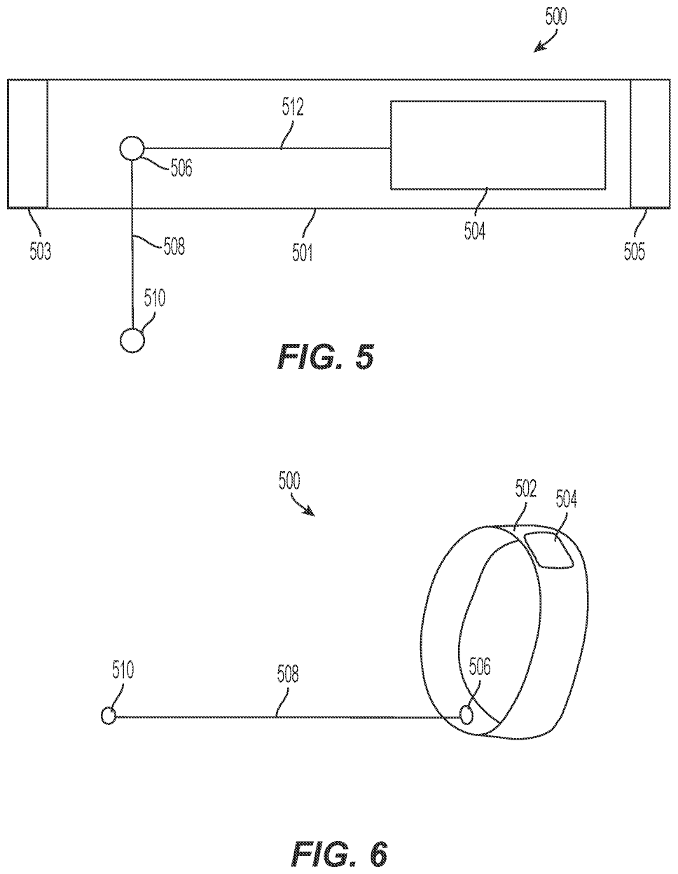

FIG. 5 shows the device 500 isolated from the patient's body in an open configuration, and FIG. 6 shows the device 500 in a closed configuration. As illustrated by FIGS. 5 and 6, the device 500 may comprise an external component in the form of a wrist band 501, an electronics component 504 carried by the band 501, an elongated conductor 508 electrically coupled to one or more components of the wrist band 501 (e.g., via one or more wires 512, fiber optic connections, etc.), and one or more sensors 510 (such as a pressure sensor and/or other sensors) electrically coupled to the elongated conductor 508 and/or electronics component 504 (directly or indirectly via an electrical connector). The band 501 may further comprise a coupler 506, and the one or more sensors 510 and/or elongated conductor 508 may be configured to be electrically coupled to the components of the band 501 via the coupler 506 (permanently or removably). The band 501 may be configured to be worn around a patient's wrist, and can include one or more coupling portions 503, 505 configured to detachably engage one another and/or another portion of the band 501 to secure the band 501 to the patient. Additionally or alternatively, the band 501 (or other external component) may comprise an adhesive element configured to secure the band 501 to the patient's skin.

As shown in FIG. 6, when the device 500 is installed in and/or on a patient with the band 501 in a closed configuration, the elongated conductor 508 can extend away from the band 501, thereby separating the sensor(s) 510 and the band 501 by a distance along the patient's arm (or other region of the patient's body where the external component is positioned). In some embodiments the elongated conductor 508 is configured to be electrically coupled directly or indirectly to the electronics component 504. When the device is installed in and/or on a patient, the band 501 may be configured to be positioned such that the electronics component is aligned with and/or positioned over a dorsal surface of the patient's wrist. The coupler 506 can be positioned such that, when the band 501 is positioned on the wrist (or other body portion), the coupler 506 is at or adjacent a location where the elongated conductor and/or catheter exits the blood vessel lumen (such as an arterial lumen).

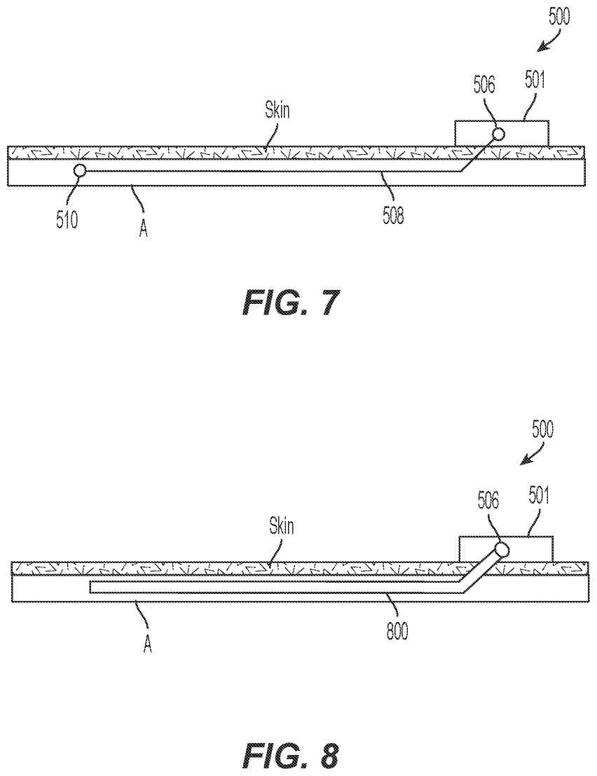

As shown in FIG. 7, when the device 500 is installed in and/or on a patient, the elongated conductor 508 can extend along and within a patient's artery A, such as a patient's radial artery or other blood vessel. As such, any sensor(s) carried by the elongated conductor 508 are configured to be disposed within the arterial lumen (or other blood vessel lumen).

As shown in FIG. 8, in some embodiments the device 500 includes a catheter 800 fixedly or removably coupled to the external component 501 and configured to extend away from the external component 501 into the patient's artery (or other blood vessel). The catheter 800 may be any of the catheters described herein. In some embodiments, the catheter 800 is an angiocatheter.

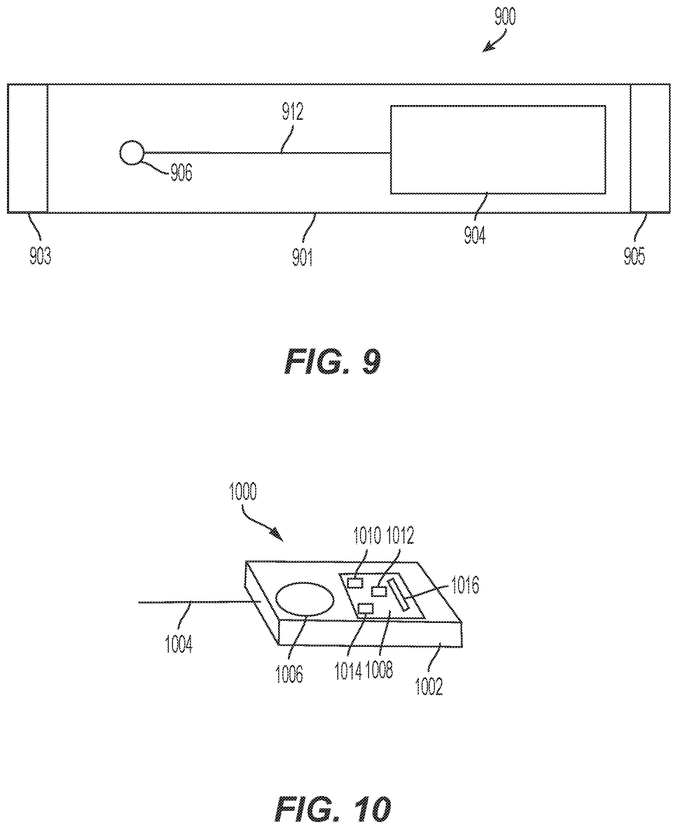

FIG. 9 is an isolated view of an external component 900 configured for use with the VAA devices disclosed herein, including the vascular monitoring devices of the present technology. The external component 900 can comprise a band 901 similar to band 501, an electronics component 904 similar to electronics component 504, coupling portions 903, 905 similar to coupling portions 503, 505, an electrical connector 912 similar to electrical connector 512, and a coupler 906 similar to coupler 506.

FIG. 10 is an isolated view of an example electronics component 1000 configured for use with the VAA devices disclosed herein, including the vascular monitoring devices of the present technology. The electronics component 1000 can include, for example, any of the electrical components detailed above, such as a controller, a multi-component semiconductor 1012, memory 1010, a data communications unit 1016 (which can include an antenna), a battery 1006, and a PCB 1008. In some embodiments, the electronics component 1000 may include more or fewer components. One, some, or all of the components may be partially or completely contained within a housing 1002. An elongated conductor 1004, such as a wire, can be fixedly or detachably coupled to the housing 1002 and/or components therein.

### CONCLUSION

Other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-10.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-13. (canceled)

14. A vascular access assembly (“VAA”), comprising:
an extension tube having a proximal end and a distal end;
a catheter having a proximal end and a distal end, the distal end configured to be positioned in a blood vessel of a patient;
a junction having an inflow region coupled to a distal end of the extension tube and an outflow region coupled to a proximal end of the catheter, wherein the junction is configured to fluidly couple the extension tube and the catheter;
a sensor configured to obtain measurements; and
at least one controller configured to be communicatively coupled to the sensor, wherein the at least one controller is further configured to—
obtain the measurements via the sensor while the catheter is positioned within the blood vessel; and
determine at least one physiological parameter based on the measurements.

15. The VAA of claim 14, wherein sensor is disposed at an interior region of the junction.

16. The VAA of claim 14, wherein the sensor is disposed at an exterior region of the junction.

17. The VAA of claim 14, wherein the sensor comprises a first sensor at an exterior region of the junction and a second sensor at an interior region of the junction.

18. The VAA of claim 14, wherein the sensor is disposed within a lumen of the extension tube.

19. The VAA of claim 14, wherein the sensor is disposed within a lumen of the catheter.

20. The VAA of claim 14, wherein the sensor is disposed within a wall of the catheter.

21. The VAA of claim 14, wherein the junction is a portion of a central venous catheter, a midline catheter, a peripherally-inserted central catheter, a peripheral intravenous line, or an arterial line catheter.

22. The VAA of claim 14, wherein at least a portion of the sensor is exposed at an exterior surface of the junction such that, when the junction is positioned against a patient's skin, the sensor is in contact with the patient's skin.

23. The VAA of claim 14, wherein the sensor comprises a plurality of sensors.

24. The VAA of claim 23, wherein each of the plurality of sensors have a portion that is exposed at an exterior surface of the junction such that, when the junction is positioned against a patient's skin, each of the sensors is in contact with the patient's skin.

25. The VAA of claim 23, wherein at least one of the sensors is disposed at an exterior surface of the junction and at least one of the sensors is disposed at an interior region of the junction.

26. The VAA of claim 14, wherein the junction is a unitary body formed of a heat molded material.

27. The VAA of claim 14, wherein the junction includes a first sensor adjacent the inflow region and a second sensor adjacent the outflow region.

28. The VAA of claim 14, wherein the junction includes an opening at the inflow region configured to receive a portion of a tubular shaft therethrough.

29. (canceled)

30. The VAA of claim 14, wherein the inflow region includes a receiving region surrounded by a body of the junction, wherein the receiving region is configured to permanently or detachably receive a portion of a tubular shaft therein.

31. (canceled)

32. The VAA of claim 14, wherein the junction is configured to be extracorporeally positioned during use.

33. The VAA of claim 14, wherein the at least one controller is configured to compare the at least one physiological parameter to a predetermined threshold.

34. The VAA of claim 33, wherein, the at least one controller is configured to provide an indication of the patient's health based on the comparison.

35. The VAA of claim 33, wherein, based on the comparison, the controller is configured to provide an indication of a health condition of a patient, the health condition being at least one of sepsis, pulmonary embolism, metastatic spinal cord compression, anemia, dehydration/volume depletion, vomiting, pneumonia, congestive heart failure, kidney failure, volume overload, performance status, arrythmia, neutropenic fever, acute myocardial infarction, pain, opioid toxicity, hyperglycemic/diabetic ketoacidosis, hypoglycemia, hyperkalemia, hypercalcemia, hyponatremia, one or more brain metastases, superior vena cava syndrome, gastrointestinal hemorrhage, immunotherapy-induced or radiation pneumonitis, immunotherapy-induced colitis, diarrhea, cerebrovascular accident, stroke, pathological fracture, hemoptysis, hematemesis, medication-induced QT prolongation, heart block, tumor lysis syndrome, sickle cell anemia crisis, gastroparesis/cyclic vomiting syndrome, hemophilia, cystic fibrosis, chronic pain, and seizure.

36-56. (canceled)
